(19) United States
(12) Patent Application Publication
(10) Pub. No.: US 2012/0004586 A1
(43) Pub. Date: Jan. 5, 2012

(54) Elbow Brace

(76) Inventor: Stephen J. Martino, Farmingdale, NJ (US)

(21) Appl. No.: 13/173,690

(22) Filed: Jun. 30, 2011

Related U.S. Application Data
(60) Provisional application No. 61/359,871, filed on Jun. 30, 2010.

(51) Int. Cl. A61F 5/00

(52) U.S. Cl. 602/20

(57) ABSTRACT

The invention is for a brace for preventing or reducing muscular and nerve disorders in the region of the elbow such as tennis elbow. The brace comprises a sleeve adapted for fitting over a wearer's arm with one end terminating above the wearer's elbow and the other end terminating below the wearer's elbow. The sleeve includes a cut-out section arranged to be located over an injured portion of the wearer's elbow joint. The brace also includes a pad which supports the elbow and has an outer contour that avoids contact with the injured area of the wearer's elbow. The pad also serves to isolate the wearer's elbow from contact and pressure.

This application claims priority to U.S. Provisional Patent Application No. 61/359,871, filed on Jun. 30, 2010. The present invention generally relates to an elastic elbow brace for treatment of injuries to the elbow joint. By isolating and protecting the cubital tunnel and the ulnar nerve which lies within, the medial epicondyle of the humerus and the surrounding soft tissue structures through cushioning, the elastic brace of the present invention is designed to treat and prevent injuries to the aforementioned areas.

### FIELD OF THE INVENTION

### Background of the Invention

Various externally applied elastic and rigid braces are in use that provide stability to the elbow and promote healing. One such common injury to the elbow is called “tennis elbow.” This term commonly applies to painful inflammation of the tendons attached to the medial or lateral epicondyle of the humerus. As a result of such things as direct trauma, repetitive use, poor elbow positioning, surgery or mechanical strain, the elbow area can become painful and limit activity.

The ulnar nerve lies in an anatomical location commonly referred to as the cubital tunnel, which is located between the medial epicondyle of the humerus and the olecranon process of the ulna bone. Through soft tissue injury of the surrounding area, trauma, repetitive use of the arm, positioning of the arm, surgery and inflammation of the tendons attached to the medial epicondyle of the humerus, the ulnar nerve can be injured. This injury could cause reversible and irreversible changes to the ulnar nerve. An ulnar nerve injury in the cubital tunnel can result in a person suffering both numbness and weakness of the hand along with weakness of some forearm muscles.

Treatments for conditions such as tennis elbow, tendonitis pertaining to the medial epicondyle of the humerus, irritation and/or inflammation of the cubital tunnel and/or surrounding tissues, and ulnar nerve injury, have usually been rather conservative. Such treatments have included rest, positioning and anti-inflammatory medication. Occasionally hydrocortisone injection, ulnar nerve transpositions, and tendon surgery have been utilized, with varying degrees of success.

Certain braces have been used to support and stabilize the elbow. For example, elastic braces are disclosed in U.S. Pat. No. 6,224,564 (Korobow) and U.S. Pat. No. 6,110,135 (Madow et al.). Both of these elastic braces work through a tubular design that surrounds the elbow and mobilizes the joint through soft and elastic material. Other examples of braces which attempt to treat the foregoing problem include an elbow brace disclosed in U.S. Pat. No. 4,441,493 (Nirschl), an elbow brace disclosed in U.S. Pat. No. 5,472,413 (Detty), and an elbow brace disclosed in U.S. Pat. No. 4,922,929 (DeJournett). These braces were designed to directly cushion the medial epicondyle of the humerus and surrounding structures. Lastly, U.S. Pat. No. 5,063,913 (Nyi) discloses an elbow brace designed with shock absorbers that fit over the medial and lateral epicondyle of the humerus.

For several reasons, the above-mentioned braces fail to adequately treat injuries to the cubital tunnel and surrounding soft tissues, ulnar nerve compression/injury in the cubital tunnel, tennis elbow (pertaining to tendonitis of the medial epicondyle of the humerus) or other similar muscular and nerve disorders in the region of the elbow. First, several of these foregoing braces apply direct and constant pressure to the injured area of the elbow. Second, several of the foregoing braces aggravate the structures of the injured area of the elbow through friction due to tightening of the brace during movement. Third, several of the foregoing braces fail to isolate the injured area of the elbow. Fourth, several of these foregoing braces do not adequately permit healing in the area because of the constant pressure applied at the injured area of the elbow. Finally, several of these foregoing braces fail to distribute pressure away from the area, thereby promoting healing.

It is an object of the present invention to provide a brace that overcomes the shortcomings of the prior art braces, such as the aforementioned braces.

It is a further object of the present invention to provide a brace that adequately treats and prevents injuries to the cubital tunnel and surrounding soft tissues, ulnar nerve compression and/or injury in the cubital tunnel, tennis elbow (specifically pertaining to tendonitis of the medial epicondyle of the humerus), and or other similar muscular and nerve disorders in the region of the elbow. The present invention would achieve this by distributing pressure, strain and friction away from injured area of the elbow by providing padding to the surrounding structures. Thereby, through selective isolation of the targeted portion of the elbow joint, the brace of the present invention will promote healing and prevent future injuries.

It is still a further object the present invention to provide a brace that is flexible, easy to handle, and can be self-applied by individuals suffering from the aforementioned problems.

It is an additional object of the present invention to provide a brace that is adaptable to various movements, is arranged to be useful in different positions, can be worn on either arm and is elastic, and flexible.

It is still an additional object of this invention to provide cushioning to the olecranon process of the ulna bone and prevent and treat injuries to this area.

### BRIEF SUMMARY OF THE INVENTION

The invention is for a brace for preventing or reducing muscular and nerve disorders in the region of the elbow such as tennis elbow. In one embodiment, the brace comprises a sleeve adapted for fitting over a wearer's arm with one end terminating above the wearer's elbow and the other end terminating below the wearer's elbow. The sleeve includes a cut-out section arranged to be located over an injured portion of the wearer's elbow joint. A pocket is formed in the sleeve for retaining a pad therein. The pad supports the elbow and has an outer contour that avoids contact with the injured area of the wearer's elbow. The pad also serves to isolate the wearer's elbow from contact and pressure.

In an alternative embodiment, the sleeve places the brace into a proper position so that the wearer can secure the brace around the joint independently with the use of a strap located above the elbow joint and another strap located below the elbow joint. The brace is padded to support the elbow. The brace is also contoured to avoid contact with the injured area of the wearer's elbow. The padding also isolates the wearer's elbow from contact and pressure. The brace may be worn on either the right or left arm.

### BRIEF DESCRIPTION OF THE DRAWINGS

FIG. 1 is an elevational view of an elbow brace of the present invention shown disposed over a wearer's elbow illustrating various anatomic aspects of the bones, and the ulnar nerve;

FIG. 2 is a cross-sectional view taken along line 2-2 of FIG. 1;

FIG. 3 is a cross-sectional view taken along line 3-3 of FIG. 1;

FIG. 4 is a cross-sectional view taken along line 4-4 of FIG. 1;

FIG. 5 is a cross-sectional view taken along line 5-5 of FIG. 1; and,

FIG. 6 is a view of the elbow pad of the elbow brace of the present invention.

FIG. 7 is an elevational view of an elbow brace of the present invention shown disposed over a wearer's elbow illustrating various anatomic aspects of the bones, and the ulnar nerve;

FIG. 7A is a fragmentary, exploded isometric view showing various layers of the brace of FIG. 7.

FIG. 8 is a cross-sectional view taken along line 8-8 of FIG. 7.

FIG. 9 is a cross-sectional view taken along line 9-9 of FIG. 7.

FIG. 10 is a cross-sectional view taken along line 10-10 of FIG. 7.

FIG. 11 is an isometric view of the elbow brace of FIG. 7 in an open position.

### DETAILED DESCRIPTION OF THE INVENTION

FIG. 1 is a view of the elbow brace 10 of the present invention. The elbow brace 10 is shown surrounding the arm 14 of a wearer. The elbow brace 10 includes a tubular sleeve 18 that may be constructed of any suitable material. For example, the sleeve 18 may be constructed from a tubular woven fabric (similar to an ACE bandage) that is flexible, provides shape and strength, is elastic, and conforms to the shape of the wearer's arm 14. The sleeve 18 may include an inner layer of hypoallergenic woven fabric material that provides a comfortable contact with the wearer's arm and will absorb perspiration. Alternatively, the sleeve 18 could also be made of fabric-covered neoprene, or a layer of Airprene® surrounded by a layer of Coolmax™ including both an inner layer 18a, and an outer layer 18b.

Referring now to FIG. 1 there is shown various anatomical aspects of the wearer's elbow joint 14, i.e., the wearer's right elbow joint, such as when the wearer's palm is facing up. The elbow joint 14 is a hinge joint and a ball and socket joint that allows for both bending and rotation. Three bones of the arm come together at the elbow joint, i.e., the humerus 22, the radius 26, and the ulna 30. The bones are connected with ligaments to keep the bones connected together and aligned to form the elbow joint 14. The humerus 22 is the bone in the upper portion of the arm that connects to the shoulder at the top and the elbow at the bottom. The humerus 22 is a long bone. As shown in FIG. 1, the humerus 22 includes a medial epicondyle at 34.

The ulna 30 is a long bone found in the forearm. The ulna 30 is the longer of the two bones found in the forearm and is the dominant bone at the elbow, allowing for flexing and extending. At the proximal end of the ulna is a c-shaped notch 32. This notch 32 is where the ulna 30 connects with the humerus 22 to form the elbow joint. Adjacent this c-shaped notch 32 is a bony projection called the olecranon process 38, which is what is felt as the point of the elbow.

A space exists between the medial epicondyle 34 and the olecranon process 38, this space being prone to compression and injury due to repetitive motion or physical activity. Such compression or injury can result in inflammation and/or injury to tendons and/or soft tissue located in proximity to this space, as well as injury to the ulnar nerve 42, which passes between the medial epicondyle 34 and the olecranon process 38. Mere inflammation of tendons in this space can result in injury to the ulnar nerve 42 which is also located therein. Such injuries are commonly referred to as tennis elbow, quarterback elbow, or pitcher's elbow.

As shown in FIG. 1, the sleeve 18 includes a generally circular cut-out section at 46 which surrounds the areas of the elbow that are prone to such injury, i.e., the medial epicondyle 34 and its surrounding soft tissue structures, along with the ulnar nerve 42 as it passes between the medial epicondyle 34, and olecranon process 38. The opening 46 is situated over these areas such that the medial epicondyle 34 is situated towards the distal end of the cut-out section 46. It is frequent that compression injury to the ulnar nerve 42 occurs a short distance, e.g., two centimeters, above the medial epicondyle 34. By positioning the medial epicondyle 34 towards the distal end of the cut-out section 46, this area of frequent compression injury is centered within the cut-out section 46.

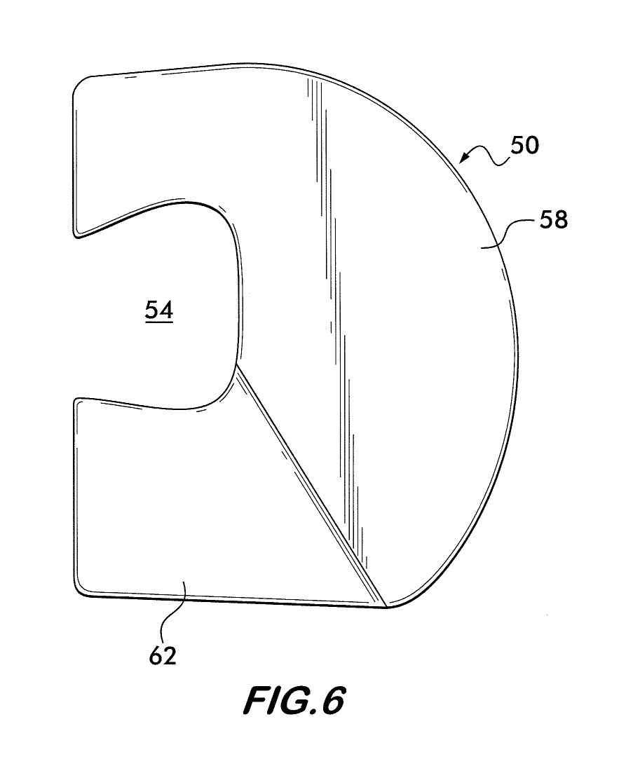

Although the cut-out section 46 of the sleeve 18 is shown as being generally circular, it should be understood that the cut-out section 46 could also be of other shapes. Referring now to FIGS. 1, 2, and 6, an elbow pad 50 is arranged to be integral with and disposed over the sleeve 18 and to wrap around the elbow joint of the wearer. As best shown in FIG. 6, the elbow pad 50 is generally in the shape of a reverse letter “C” and includes an open carved-out section 54 that follows the contour of a portion of the cut-out section 46. In this manner, if the cut-out section 46 of the sleeve 18 is round, then the carved-out section 54 of the elbow pad 50 would be round. Likewise, if the cut-out section 46 of the sleeve 18 is D-shaped, then the carved-out section 54 of the elbow pad 50 will be similarly shaped. In this manner, the elbow pad 50 surrounds and avoids contact with the injured area of the elbow joint located inside the cut-out section 46, that area including the medial epicondyle 34 and its surrounding soft tissue structures, along with the ulnar nerve 42 as it passes between the medial epicondyle 34 and olecranon process 38. Due to its thickness, the elbow pad 50 serves to isolate these structures from contact, pressure, and friction with surfaces.

Referring now to FIGS. 1, 5, and 6, the elbow pad 50 includes a thinner padded section 58 and a thicker padded section 62. As best shown in the figures, the thinner padded section 58 extends distally from the proximal end of the elbow brace 10 and wraps around the upper and side portions of the cut-out section 46. The thicker padded section 62 extends across the lower portion of the carved-out section 54 and serves to further isolate from contact with surfaces the injured areas of the elbow joint located inside the cut-out section 46, such as the medial epicondyle 34 and its surrounding soft tissue structures, along with the ulnar nerve 42 as it passes between the medial epicondyle 34 and olecranon process 38. The thinner and thicker padded sections 58, 62 can be of any suitable thickness. For example, the thinner padded section 58 could be of a thickness of 0.75 inches while the thicker padded section 62 could be of a thickness of 1.5 inches. The pad 50 can be formed of any suitable cushioning material, including Conforfoam®, Poly-Fil NU-FOAM™, natural lambskin, foam, polymeric foam or polyester sponge material. The purpose for providing a thicker padded section 62 is to further isolate the anatomic structures discussed above from contact, pressure, and friction.

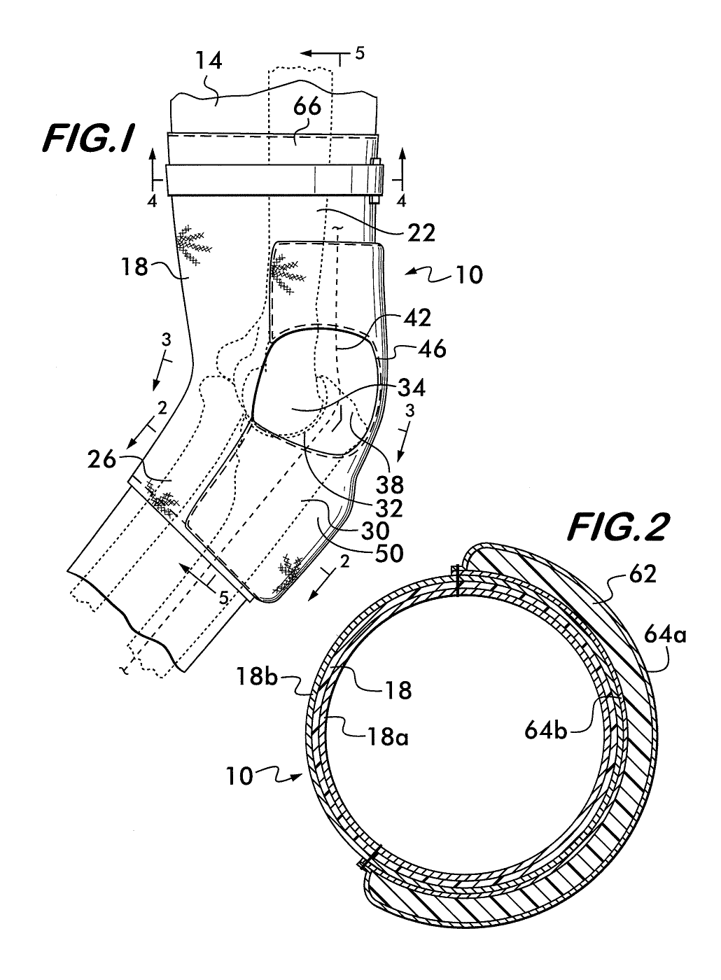

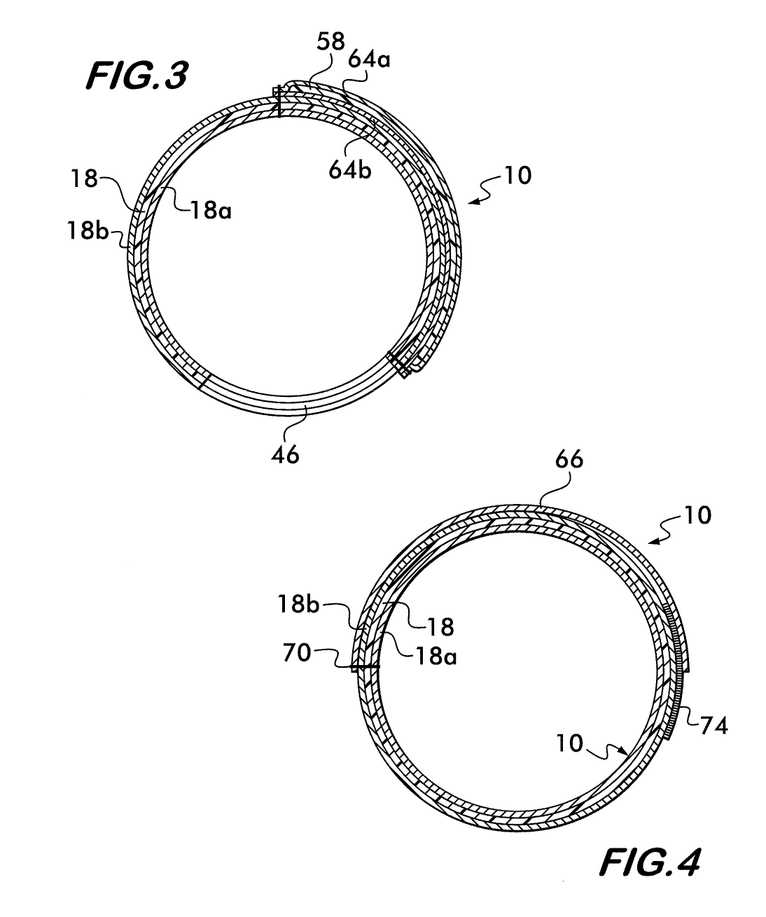

As best shown in FIGS. 2 and 5, the pad 50 is situated within a fabric pocket formed of an outer layer 64a that is sewn to an inner layer 64b, the inner layer 64b being sewn to the outside of the sleeve 18. The fabric forming the envelope may be formed of any suitable material. The pocket is shaped and arranged for snugly fitting the pad 50 therein.

By arranging the elbow pad 50 to surround at least a portion of the cut-out section 46, no matter how the wearer positions his or her arm, especially during sleeping, the injured area of the elbow within the cut-out section 46 remains isolated from contact and friction. As the arm is flexed, extended, pronated, or supinated, there is no contact by the elbow brace 10 with the area within the cut-out section 46 that will cause friction or rubbing on the injured portion of the elbow. The elbow pad 50 wraps around the medial epicondyle 34 and the olecranon process 38, this area being prone to compression and injury due to repetitive motion or physical activity. The elbow pad 50 also wraps around the ulnar nerve 42 as it passes between the medial epicondyle 34 and the olecranon process 38, which is also an area prone to injury. The elbow pad 50 can flex and extend in accordance with the natural movements of the elbow and is woven into place onto the elbow brace. By reducing such pressure, strain and tension in the injured area of the elbow, healing will be promoted.

The ends of the sleeve 18 and the cut-out section 46 may be hemmed or finished as any other conventional fabric. The elbow brace 10 may also have its outer surface colored or decorated as desired.

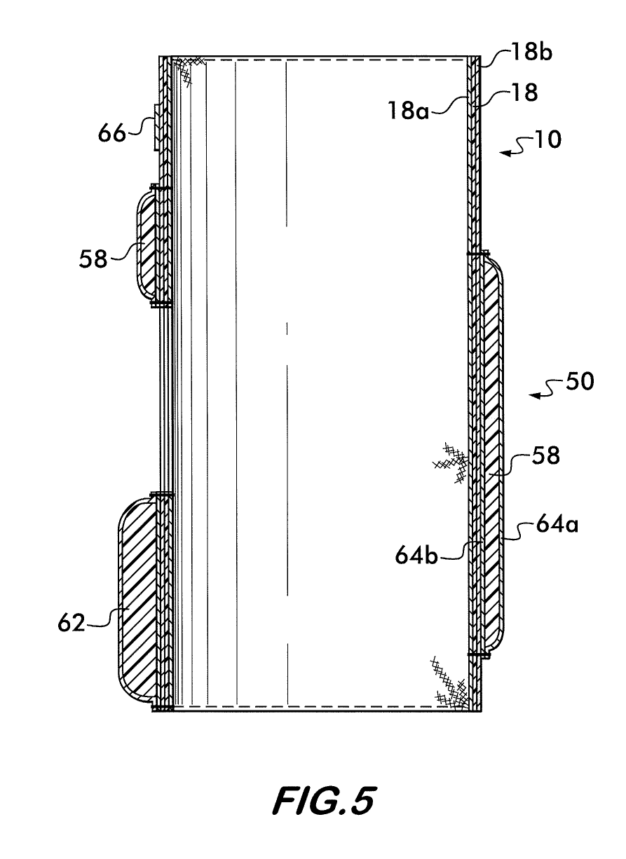

Referring now to FIGS. 1 and 4, a wrap-around strap 66 is located on the upper portion of the elbow brace 10. As best shown in FIG. 4, the wrap-around strap 66 includes a fixed end 70 that is affixed to the elbow brace 10 by any suitable means, e.g., sewing. At its opposite end, the wrap-around strap 66 is free and is provided with a flexible hook component of a VELCRO® fastening system or other equivalent fastening system. A cooperating loop component, indicated at 74, is fastened, e.g., sewn, in place on the elbow brace 10 in a conventional manner. In this manner, the wrap around support 66 can be wrapped tightly about the elbow brace 10 above the wearer's elbow to ensure that the elbow brace 10 is secured to the wearer's arm and won't slip down during use. When applying the elbow brace 10, the wearer simply extends his hand and arm through the sleeve 18 so that the brace 10 is positioned radially about the wearer's arm 14 and the medial epicondyle 34 is situated distally to the medially located carved-out section 54 of the sleeve 18.

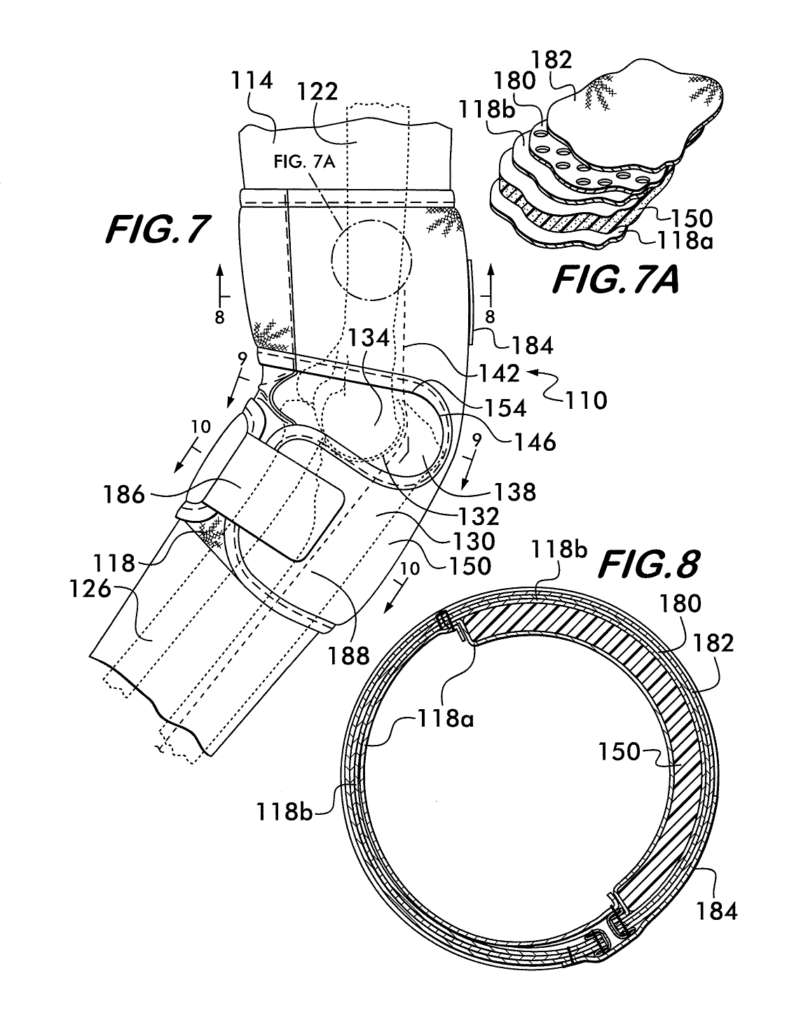

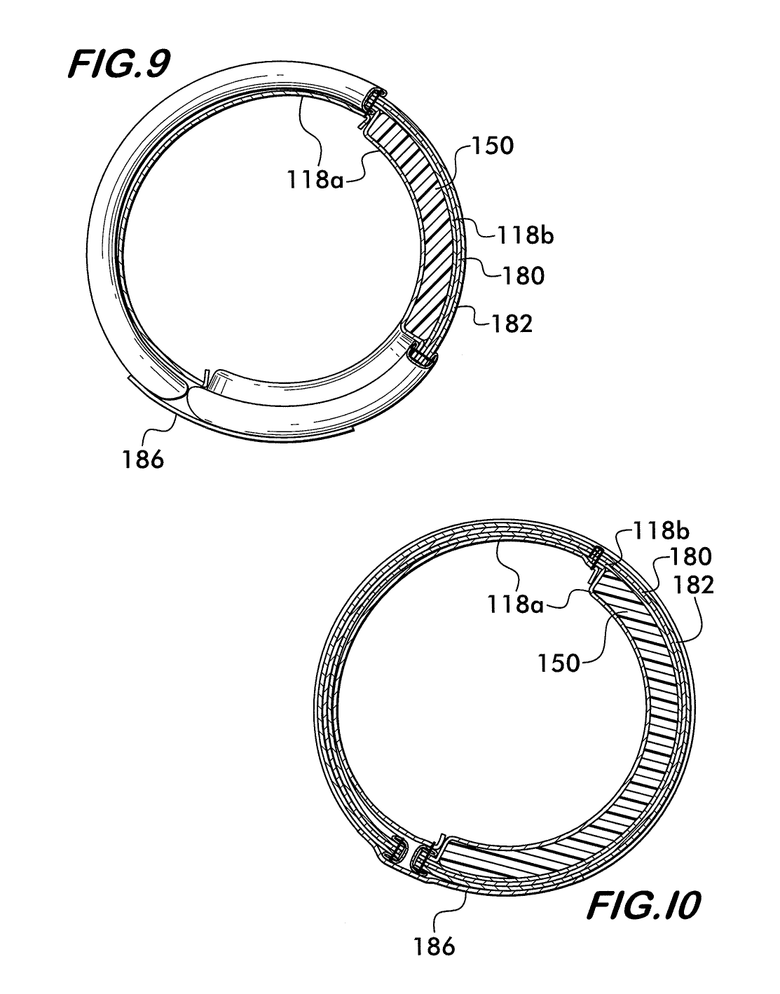

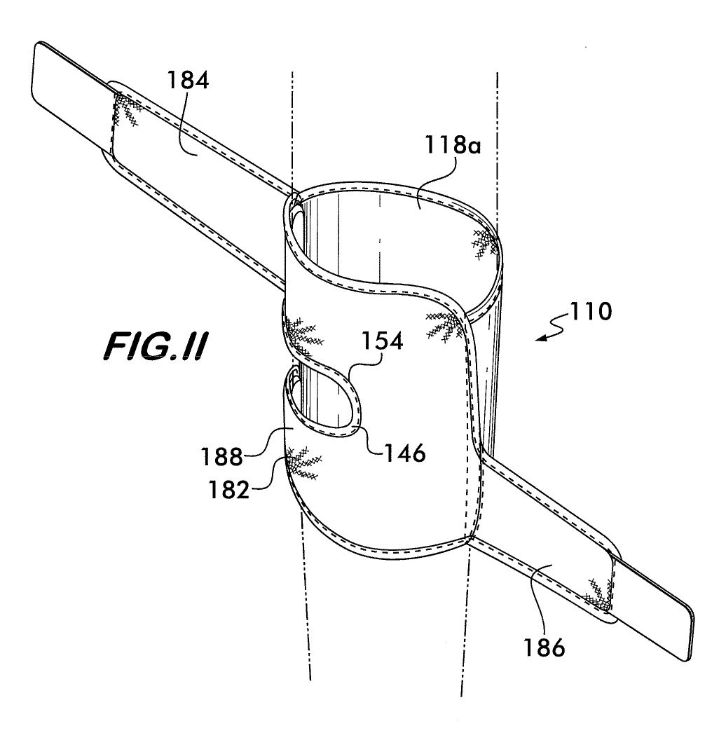

An alternative embodiment of an elbow brace 110 of the present invention is shown in FIGS. 7-11.

FIG. 7 is a view of the elbow brace 110 of the present invention. The elbow brace 110 is shown surrounding the arm 114 of a wearer. The elbow brace 110 includes a sleeve 118 that may be constructed of any suitable material, e.g., from a woven fabric (similar to an ACE bandage) that is flexible, provides strength and conforms to the shape of the wearer's arm 114. The brace 110 may include an inner layer of hypoallergenic woven fabric material that provides a comfortable contact with the wearer's arm and will absorb perspiration. Alternatively, the sleeve 118 could also be made of fabric-covered neoprene, or a layer of Airprene® surrounded by a layer of Coolmax™. The sleeve 118 of FIGS. 7-11 preferably includes an inner layer 118a and an outer layer 118b. In one embodiment, the brace 110 may include light pre-shaped hinges (not shown) along the lateral side that can provide enhanced support to the brace 110. The hinges would preferably be positioned along a wearer's lateral epicondyle to add stability and aid in mobility of the brace 110.

FIG. 7 shows various anatomical aspects of the wearer's elbow joint 114, i.e., the wearer's right elbow joint, such as when the wearer's palm is facing up. Although FIG. 7 shows the wearer's right elbow joint, the elbow brace 110 is preferably usable on both of the wearer's arms.

The elbow joint 114 is a hinge joint and a ball and socket joint that allows for both bending and rotation. Three bones of the arm come together at the elbow joint, i.e., the humerus 122, the radius 126, and the ulna 130. The bones are connected with ligaments to keep the bones connected together and aligned to form the elbow joint 114. The humerus 122 is the bone in the upper portion of the arm that connects to the shoulder at the top and the elbow at the bottom. The humerus 122 is a long bone. As shown in FIG. 7, the humerus 122 includes a medial epicondyle at 134.

The ulna 130 is a long bone found in the forearm. The ulna 130 is the longer of the two bones found in the forearm and is the dominant bone at the elbow, allowing for flexing and extending. At the proximal end of the ulna is a c-shaped notch 132. This notch 132 is where the ulna 130 connects with the humerus 122 to faun the elbow joint. Adjacent this c-shaped notch 132 is a bony projection called the olecranon process 138, which is what is felt as the point of the elbow.

A space exists between the medial epicondyle 134 and the olecranon process 138, this space being prone to compression and injury due to repetitive motion or physical activity. Such compression or injury can result in inflammation and/or injury to tendons and/or soft tissue located in proximity to this space, as well as injury to the ulnar nerve 142, which passes between the medial epicondyle 134 and the olecranon process 138. Mere inflammation of tendons in this space can result in injury to the ulnar nerve 142 which is also located therein. Such injuries are commonly referred to as tennis elbow, quarterback elbow, or pitcher's elbow.

Referring now to FIG. 11, there is shown an isometric view of the elbow brace of FIG. 7 in an open position. As shown in FIGS. 7 and 11, the brace 110 includes a cut-out section at 146 which surrounds the areas of the elbow that are prone to such injury as discussed above, i.e., the medial epicondyle 134 and its surrounding soft tissue structures, along with the ulnar nerve 142 as it passes between the medial epicondyle 134, and olecranon process 138. The cut-out section 146 is situated over these areas such that the medial epicondyle 134 is situated towards the distal end of the cut-out section 146. It is frequent that compression injury to the ulnar nerve 142 occurs a short distance above and below the medial epicondyle 134. By positioning the medial epicondyle 134 towards the distal end of the cut-out section 146, this area of frequent compression injury is centered within the cut-out section 146. Although the cut-out section 146 of the brace 110 is shown as being generally oval or c-shaped, it should be understood that the cut-out section 146 could also be of other shapes.

As shown in FIGS. 7-11, a pad 150 is integral with the brace 110. The pad 150 is sandwiched between the inner layer 118a and outer layer 118b of the sleeve 118. The pad 150 is adapted to wrap around the elbow joint of the wearer. The pad 150 includes an open carved-out section 154 that follows the contour of a portion of the cut-out section 146. Thus, for example, since the cut-out section 146 of the sleeve 118 is oval or c-shaped, the carved-out section 154 of the pad 150 is likewise oval or c-shaped. In this manner, the pad 150 surrounds and avoids contact with the injured area of the elbow joint located inside the cut-out section 146, that area including the medial epicondyle 134 and its surrounding soft tissue structures, along with the ulnar nerve 142 as it passes between the medial epicondyle 134 and olecranon process 138. Due to its thickness, the pad 150 serves to isolate these structures from contact, pressure, and friction with surfaces.

The pad 150 may be of a thickness of, e.g., 0.75 inches, but other thicknesses may also suffice. The pad 150 can be formed of any suitable cushioning material, including Confor® foam, Poly-Fil NU-FOAM™, natural lambskin, foam, polymeric foam or polyester sponge material.

FIG. 7A is a fragmentary, exploded isometric view showing various layers of the brace of FIG. 7. As shown in FIGS. 7A-10, and as described above, the pad 150 is sandwiched within the sleeve 118, i.e., between the inner layer 118a and outer layer 118b. A layer of perforated, breathable material 180 is situated on top of the outer layer 118b of the sleeve 118. Finally, an outer brace layer 182 is situated on top of the layer of perforated, breathable material 180.

By arranging the pad 150 and its carved-out section 154 to follow the contour of a portion of the cut-out section 146, no matter how the wearer positions his or her arm, especially during sleeping, the injured area of the elbow within the carved-out section 154 remains isolated from contact and friction. As the arm is flexed, extended, pronated, or supinated, there is no contact by the elbow brace 110 with the area within the carved-out section 154 that will cause friction or rubbing on the injured portion of the elbow. The pad 150 wraps around the medial epicondyle 134 and the olecranon process 138, this area being prone to compression and injury due to repetitive motion or physical activity. The pad 150 also wraps around the ulnar nerve 142 as it passes between the medial epicondyle 134 and the olecranon process 138, which is also an area prone to injury. The pad 150 can flex and extend in accordance with the natural movements of the elbow. By reducing such pressure, strain and tension in the injured area of the elbow, healing will be promoted.

The ends of the sleeve 118 and the carved-out section 154 may be hemmed or finished as any other conventional fabric. The elbow brace 110 may also have its outer surface colored or decorated as desired.

As shown in FIGS. 7 and 11, the elbow brace 110 includes an upper strap 184 and a lower strap 186. The upper strap 184 is adapted to wrap around the wearer's arm above the elbow joint while the lower strap 186 is adapted to wrap around the wearer's arm below the elbow joint. The free ends of both straps 184,186 preferably include a VELCRO® (hook or loop) surface or equivalent fastening means adapted to secure the straps 184, 186 to the outer brace layer 182 when the elbow brace 110 is secured to the wearer, as shown in FIG. 7. The outer brace layer 182 preferably comprises a surface 188 adapted to secure the VELCRO® surface (or equivalent fastening means) of the free ends of the straps 184,186.

When applying the elbow brace 110, the wearer simply extends his hand and arm through the sleeve 118 so that the brace 110 is positioned radially about the wearer's arm 114 and the medial epicondyle 134 is situated distally to the medially located cut-out section 146 of the sleeve 118. The wearer then wraps the straps 184,186 around the brace 110, thereby comfortably securing the brace 110 about the wearer's elbow joint 114.

It is understood that the elbow brace and its constituent parts described herein is an exemplary indication of a preferred embodiment of the invention, and is given by way of illustration only. In other words, the concept of the present invention may be readily applied to a variety of preferred embodiments, including those disclosed herein. While the invention has been described in detail and with reference to specific examples thereof, it will be apparent to one skilled in the art that various changes and modifications can be made therein without departing from the spirit and scope thereof.

I claim:

1. A brace for preventing or reducing tennis elbow and similar muscular and nerve disorders in the region of the elbow, said brace comprising:
a. a sleeve adapted for fitting over a wearer's arm with one end of said sleeve terminating above the wearer's elbow and the other end of said sleeve terminating below the elbow, said sleeve including a cut-out section having a contour and located over a region of the wearer's elbow including the medial epicondyle, olecranon process, and the space therebetween;
b. a pocket formed in said sleeve and extending from above the elbow to below the elbow; and,
c. a pad enclosed within said pocket so as to be carried by said sleeve, said pad supporting the elbow and having an outer contour that follows at least a portion of said cut-out section contour to isolate the wearer's elbow from contact and pressure.

2. The brace as defined in claim 1, additionally comprising a strap provided for attaching said sleeve to the arm of the wearer, said strap adapted to wrap around the arm of the wearer.

3. The brace as defined in claim 1, wherein the cut-out section is generally circular or oval in shape and wherein said elbow pad includes an upper portion, a lower portion, and a carved-out portion therebetween, said carved-out portion following the contour of a portion of the cut-out portion.

4. The brace as defined in claim 2, wherein said strap comprises one of the group of flexible hook and loop fastener strips, and flexible straps with buckle fasteners.

5. The brace as defined in claim 1, wherein said pad is formed of a material that is capable of absorbing shock energy.

6. The brace as defined in claim 1, wherein said sleeve is woven of an elastic fabric.

7. The brace as defined in claim 1, wherein said sleeve is tubular and flexible.

8. The brace as defined in claim 1, wherein said sleeve includes an arm engaging layer to produce comfort and improve frictional engagement with the skin of the wearer.

9. The brace as defined in claim 1, wherein said pad can flex and extend in accordance with the natural movements of the elbow.

10. The brace as defined in claim 1, wherein said pad is formed of a polymeric foam or polyester sponge material.

11. The brace as defined in claim 2, wherein said strap includes a first end affixed to said sleeve by sewing, and a free end.

12. The brace as defined in claim 11, wherein a surface of a hook and loop fastening system is located at the free end of said strap, said surface being arranged to fasten to a complementary surface on said sleeve.

13. The brace as defined in claim 1, wherein said sleeve is constructed of a hypoallergenic material.

14. A method for treating tennis elbow and similar muscular and nerve disorders in the region of the elbow, comprising the steps of:
a. providing a brace to a wearer, said brace comprising a sleeve adapted for fitting over the wearer's arm with one end of said sleeve terminating above the wearer's elbow and the other end of said sleeve terminating below the elbow, said sleeve including a cut-out section having a contour, said brace further comprising a pad adapted to wrap around the wearer's elbow, said pad including a carved-out section that follows said contour of a portion of said cut-out section; and
b. positioning the cut-out section over a region of the wearer's elbow including the medial epicondyle, olecranon process, and the space therebetween.

15. The method as defined in claim 14, wherein said brace is adapted to be worn on both a wearer's right arm and left arm.

16. The method as defined in claim 14, said brace further comprising an upper strap and a lower strap, said method further comprising the steps of wrapping said upper strap around a portion of the wearer's arm above the elbow and wrapping said lower strap around a portion of the wearer's arm below the elbow, thereby securing said brace to the wearer's arm.

17. The method as defined in claim 14, wherein said pad is sandwiched between an inner layer and an outer layer of said sleeve.

18. The method as defined in claim 14, wherein the cut-out is generally oval in shape.

19. An elbow brace comprising a sleeve adapted for fitting over a wearer's arm wherein one end of said sleeve is adapted to terminate above a wearer's elbow and the other end of said sleeve is adapted to terminate below a wearer's elbow, said sleeve including a cut-out section having a contour, the cut-out section being located along a side portion of the sleeve, said brace further comprising a pad adapted to wrap around a wearer's elbow, said pad including a carved-out section that follows said contour of a portion of said cut-out section.

20. The elbow brace as defined in claim 19 further comprising an upper strap and a lower strap, said upper strap adapted for wrapping around a portion of a wearer's arm above the elbow, said lower strap adapted for wrapping around a portion of a wearer's arm below the elbow in order to secure said brace to a wearer's arm.
